(19) United States
(12) Plant Patent Application Publication
(10) Pub. No.: US 2015/0296676 P1
(43) Pub. Date: Oct. 15, 2015

(54) Fuchsia plant named 'FWURB19-0'

(50) ;

(71) Applicant: Hubertus Volmary, Munster (DE)

(72) Inventor: Hubertus Volmary, Munster (DE)

(21) Appl. No.: 13/999,984

(22) Filed: Apr. 11, 2014

(51) Int. Cl. A01H 5/00

(52) U.S. Cl. PLT/300

(57) ABSTRACT

A new and distinct Fuchsia cultivar named ‘FWIRP26-0’ is disclosed, characterized a compact, semi-trailing plant habit, with a large number of flowers, with at least 2 flowers at every axil. Flowers are pendulous and have a bright pink calyx, violet corolla, and flowering response is not dependent upon day length. The new variety is a Fuchsia, typically produced as a garden or container plant.

### LATIN NAME OF THE GENUS AND SPECIES

Fuchsia hybrid

### VARIETY DENOMINATION

‘FWIRP26-0’

### BACKGROUND OF THE INVENTION

The new cultivar is a product of a planned breeding program. The new variety originated from a cross pollination of the seed parent, an unnamed, unpatented proprietary selection of Fuchsia hybrid with the pollen parent a different unnamed, unpatented Fuchsia hybrid. The crossing was made during the Summer of 2009, at a research greenhouse in Munster, Germany. ‘FWIRP26-0’ was discovered by the inventor. Hubertus Volmary, a citizen of Germany, at the same research greenhouse during the Spring of 2010.

Asexual reproduction of the new cultivar ‘FWIRP26-0’ was first performed in Munster Germany, at a commercial greenhouse by vegetative cuttings in Summer of 2010. ‘FWIRP26-0’ has since produced several generations and has shown that the unique features of this cultivar are stable and reproduced true to type.

### SUMMARY OF THE INVENTION

The cultivar ‘FWIRP26-0’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘FWIRP26-0’. These characteristics in combination distinguish ‘FWIRP26-0’ as a new and distinct Fuchsia cultivar:

1. Compact and semi-trailing plant habit.

2. Highly floriferous plants.

3. Flowering response begins mid season

4. Distinctive intense flower coloration; bright pink calyx with violet corolla.

5. Suitability for commercial basket production

6. Flowers pendulous.

7. 2 or more flowers emerging from each axil.

8. Double flowers

### PARENT COMPARISON

Plants of the new cultivar ‘FWIRP26-0’ are similar to plants of the seed parent, in most horticultural characteristics; however, plants of the new cultivar differ in the following:

1. Parent variety plants are not as well branched.

2. Parent variety has pale pink and violet flowers.

3. Seed parent has darker branch stems.

4. Seed parent has darker green foliage.

5. Parent variety grows more vigorously.

Plants of the new cultivar ‘FWIRP26-0’ are similar to plants of the pollen parent, in most horticultural characteristics; however, plants of the new cultivar differ in the following:

1. Pollen parent variety plants are upright.

2. Parent variety has red and white flowers.

3. Pollen parent has lighter green leaves

4. Pollen parent has single flowers.

5. Parent variety ha smaller flowers

### COMMERCIAL COMPARISON

Plants of the new cultivar ‘FWIRP26-0’ are best compared to the commercial variety Fuchsia ‘Kiefuzak’ U.S. Plant Pat. No. 13,764. The two Fuchsia varieties are similar in most horticultural characteristics: however, the new variety differs in the following ways:

1. Comparison variety has an upright plant habit, whereas the new variety has a semi-trailing growth habit

2. Comparison variety flowers of a some different color, and with less intensity of color.

3. Comparison variety has larger flowers.

4. Comparison variety produces less flowers per plant.

5. Comparison variety has strong vigor whereas candidate variety has medium vigor.

### BRIEF DESCRIPTION OF THE PHOTOGRAPH

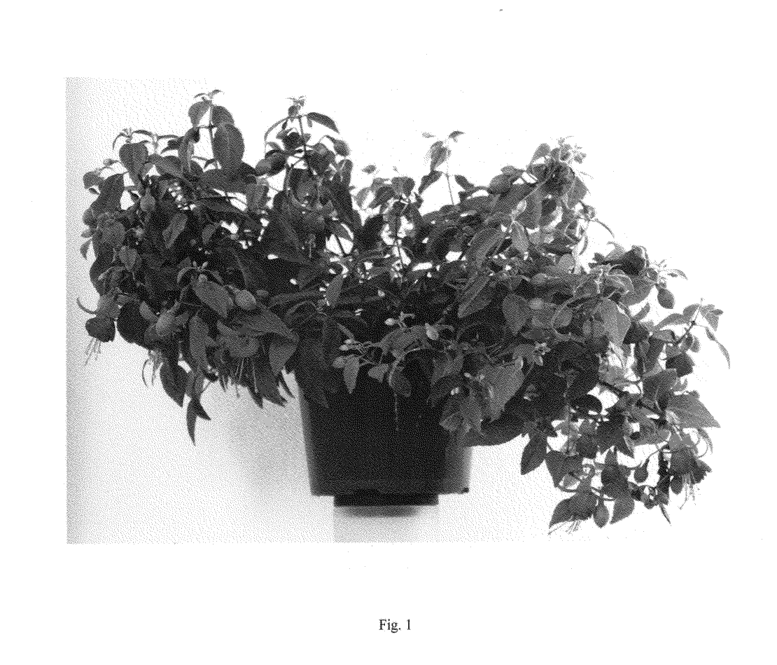

The accompanying photograph in FIG. 1 illustrates in full color a typical plant of ‘FWIRP26-0’ grown outdoors in Santa Paula, Calif. This plant is approximately 30 weeks old, shown in a 10 inch pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Colour Chart, 2007, except where general terms of ordinary dictionary significance are used. The following observations and measurements describe ‘FWIRP26-0’ plants in a commercial greenhouse in Santa Paula, Calif. Temperatures ranged from 10° C. to 29° C. night and day. No artificial light, photoperiodic treatments or chemical treatments were given to the plants. Natural light conditions were approximately 2500 to 4000 fc of light. Measurements and numerical values represent averages of typical plant types.
Botanical classification: Fuchsia hybrid ‘FWIRP26-0’
Propagation:
   Propagation method.—Vegetative Cuttings.
   Time to initiate roots.—About 2 weeks at 15° C. to 22° C.
   Root description.—Fine, densely fibrous.
Plant:
   Age of plant described.—Approximately 17 weeks from 3 rooted cuttings.
   Pot size of plant described.—10 inch.
   Height.—Approximately 27 cm from soil line of pot to top of foliar and uppermost flowering plane.
   Plant spread.—Approximately 58 cm.
   Growth rate.—Rapid.
   Growth habit.—Semi-trailing, compact.
   Branching characteristics.—Free branching.
   Length of primary lateral branches.—Average 30 cm.
   Diameter of lateral branches.—Approximately 0.3 cm.
   Quantity of primary lateral branches.—Approximately 15.
   Characteristics of primary lateral branches.—Color: Near RHS Yellow-Green 144C, flushed Greyed-purple 185B Texture: Glabrous. Strength: Strong, flexible.
   Internode length.—Average range 2 cm to 4 cm.
Foliage:
   Leaf.—Arrangement: Opposite. Quantity: Approximately 8 to 12 fully expanded leaves per primary lateral branch. Average Length: 5 cm including petiole. Average Width: 2.2 cm. Shape of blade: Ovate. Apex: Acute Base: Oblique. Margin: Shallowly denate. Texture of top surface: Glabrous. Texture of bottom surface: Glabrous. Aspect: Slight downward curve. Color: Young foliage upper side: Near RHS Green 137A Young foliage under side: Near RHS Green 138A Mature foliage upper side: Near RHS Green 137A, but darker Mature ,foliage under side: Near RHS Green 137C Venation: Type: Pinnate. Venation color upper side: Near RHS Green Group 143D Venation color under side: Near RHS Green Group 143A Attachment: Stalked. Petiole: Length: Average 1.7 cm. Diameter: Average .15 cm. Texture: Glabrous Color: Upper surface: Near RHS Yellow-Green 144C Lower surface: Near RHS Yellow-Green 144C.
Flower:
   Natural flowering season.—Flowering begins around April and continues until October.
   Inflorescence and flower type and habit.—At least 2 flowers emerge singly from each axil. Flowers emerge at different times, resulting in flowers of different maturity at each axil. Flowers are symmetrical and composed of a corolla surrounded by a decorative fused calyx. Flowers are pendulous.
   Flowers per plant.—Approximately 90 fully open flower and 450 buds on a mature plant.
   Flower longevity on plant.—Approximately 1 week for individual flowers.
   Persistent or self-cleaning.—Self-cleaning.
   Flower size (petals and sepals only, not including protruding stamen and style).—Length: Approximately 3 cm Width: Approximately 6.7 cm.
   Bud.—Shape: Oblong, with an inflated sphere at terminal end. Length: 4.2 cm Diameter: 2.3 cm Color: Near RHS Red 46C with very shiny appearance.
   Corolla.—.
   Petals (Measured from separation at top of tube).—Number: Average 7 Length: Approximately 1.8 cm Width: Approximately 2.2 cm Shape: Obovate Aspect: Slightly inwardly cupped or slightly undulating Margin: Entire. Texture: Glabrous, all surfaces. Appearance: Matte. Apex: Obtuse.
   Color.—When opening: Upper surface: Near RHS Violet 83A mid vein near Red-Purple N57A, basal blotch colored Red-Purple 58C and 58D Lower surface: Near RHS Violet 83A, mid vein near Red-Purple N57A, basal blotch colored Red-Purple 58C and 58D Fully opened: Upper surface: Near RHS, Purple N78A mid vein near Red-Purple 57A, basal blotch colored near Red 52D Lower surface: Near RHS, Purple N78A mid vein near Red-Purple 57A, basal blotch colored near Red 52D Fading: Upper surface: Near RHS, Purple N78A, but darker, mid vein, near Red-Purple N57A, basal blotch colored near Red 52D. Lower surface: Near RHS Purple N78A, but darker, mid vein, near Red-Purple N57A, basal blotch colored near Red 52D.
   Calyx.—Form: Funnelform base, separating approximately 20% from base, to flare out into 4 large decorative deltoid shaped sepals. Sepals strongly reflexed back. Fused Section: Length: Approximately 0.6 cm Diameter: Approximately 0.6 cm Unfused Section: Length: Approximately 3.0 cm Diameter: Approximately 1.2 cm Sepal texture: Glabrous all surfaces. Appearance: Iridescent. Apex: Acute. Color: Upper Surface: Near RHS Red 52A, but much brighter, Lower Surface: Near RHS Red 52A, but much brighter.
   Fragrance.—None.
Reproductive organs:
   Stamens.—Number (Per flower): Typically 8 Filament length: Average range from 2.0 cm to 3.2 cm Filament color: Near RHS Red-Purple N57A Anthers: Shape: Globular Length: Approximately 0.3 cm. Color: Near RHS Grey-Purple 187C Pollen: Amount: Scarce, colored Orange-White 159C.
   Pistils.—Quantity per flower: 1 Length: Approximately 5 cm Style: Length: Approximately 6.3 cm Color: Red-Purple N57A Stigma: Shape: Globular Color: Near Greyed-Purple 187A Length: Approximately 0.3 cm Diameter: Approximately 0.3 cm Ovary: Length: Approximately 0.6 cm Diameter: Approximately 0.4 cm Color: Near RHS Green 143C.
Other characteristics:
   Seeds and fruits.—Not observed to date.
   Disease/pest resistance.—Neither resistance nor susceptibility to normal diseases and pests of Fuchsia has been observed.
   Temperature tolerance.—Tolerates temperatures from approximately −1 C. to 38 C.
   Drought tolerance.—No tolerance for drought.

What is claimed is:

1. A new and distinct cultivar of Fuchsia plant named ‘FWIRP26-0’as herein illustrated and described.
